(19) United States
(12) Patent Application Publication
(10) Pub. No.: US 2026/0000521 A1
(43) Pub. Date: Jan. 1, 2026

(54) LOCKING FIXATION DEVICE

(71) Applicant: ATLAS SPINE, INC., Jupiter, FL (US)

(72) Inventor: Matthew G. Baynham, Jupiter, FL (US)

(21) Appl. No.: 19/322,106

(22) Filed: Sep. 8, 2025

Related U.S. Application Data
(63) Continuation-in-part of application No. 18/614,027, filed on Mar. 22, 2024.

(51) Int. Cl. A61F 2/44; A61F 2/30

(52) U.S. Cl. CPC A61F 2/447; A61F 2/30749; A61F 2002/30459; A61F 2002/30507

(57) ABSTRACT

A locking fixation device formed from a malleable wedge-shaped implant adapted to be applied to the human spine for fusing vertebral bodies together. The present invention is defined by an insert end spaced apart from a spacer end by first and second side members, each cut by recesses of uniform thickness forming a sinusoidal pattern. The fixation device includes first and second receiving holes adapted to receive bone screws for securing to vertebral bodies. A locking member rotatably secured to the spacer end includes tabs constructed and arranged to rotate from an installation position allowing insertion of the bone screws to a locking position to prohibit bone screw movement. In some embodiments, a staple member is coupled to the spacer end between a locking screw and the locking member, the staple member including an aperture and a pair of tines extending diagonally to penetrate bone and provide fixation.

### PRIORITY CLAIM

In accordance with 37 C.F.R. 1.76, a claim of priority is included in an Application Data Sheet filed concurrently herewith. Accordingly, the present invention claims priority as a continuation-in-part of U.S. patent application Ser. No. 18/614,027, entitled “LOCKING FIXATION DEVICE”, filed Mar. 22, 2024. The contents of the above referenced applications are incorporated herein by reference in its entirety.

### FIELD OF THE INVENTION

The present invention relates generally to surgically-implantable spinal devices and, more specifically, to a locking fixation device formed from a spinal implant with a malleable body.

### BACKGROUND OF THE INVENTION

The human spine contains several types of joints that contribute to its stability, flexibility, and overall function of the spine. Facet joints (Zygapophyseal Joints) connect adjacent vertebrae. Synovial joints have a membrane that produces synovial fluid, facilitating smooth movement between the articular surfaces of the vertebrae. These facet joints play a crucial role in guiding and limiting the movement of the spine, including flexion, extension, rotation, and lateral bending.

Intervertebral discs are fibrocartilaginous structures located between adjacent vertebrae. The intervertebral discs serve as shock absorbers, providing cushioning and support to the spine during various activities. Intervertebral discs contribute to the flexibility and mobility of the spine. The sacroiliac joint (SI Joint) is located at the junction of the sacrum and ilium in the pelvis. While not directly on the back of the spine, the SI joint plays a significant role in the stability and movement of the lower spine and pelvis. It primarily absorbs shock and transfers forces between the upper body and the legs during weight-bearing activities.

Atlanto-occipital Joint and Atlanto-axial Joint are located between the skull and the first cervical vertebra (C1, the atlas), and between the first and second cervical vertebrae (C1 and C2, the axis), respectively. They contribute to the mobility and stability of the neck and head. These various joints in the back of the spine work together to support the body, facilitate movement, and protect the spinal cord while allowing for flexibility and mobility in different regions of the spine. Dysfunction or injury to these joints can lead to pain, stiffness, and reduced mobility in the spine and surrounding areas.

There are numerous implants and associated methods for performing stabilization and/or immobilization in the spine. Conventional implants utilize bone screws that are threaded through the superior and inferior facets to immobilize the facet joint so as to permit the adjoined bone sections to fuse together.

U.S. Pat. No. 9,629,727 discloses a facet joint fixation device formed from a wedge shaped implant constructed from a single piece of material where each side support is cut by a series of recesses.

What is needed is a malleable fixation device that implements an interlocking component to prevent bone screws from backing out.

While existing fixation devices employing screws and locking members can provide stabilization of the sacroiliac joint, there remains a need for supplemental features to address forces at the spacer end of the implant. For example, an anchoring member, such as a staple, may be configured to provide additional fixation and resist migration or rotational forces.

### SUMMARY OF THE INVENTION

Embodiments of the invention are directed, inter alia, to a fixation device that consists of a malleable wedge-shaped implant anchored by a pair of bone screws. The fixation device is constructed from a single piece of material having an insert end spaced apart from an outer spacer end by first and second arcuate side members. Each side support is cut in a sinusoidal or accordion shape, retaining thickness, to provide spring-type resilience and permit access from unobstructed angles.

In some embodiments, the spacer end further includes a staple member secured thereto. The staple member comprises an aperture aligned with a receiving hole in the spacer end for receiving a locking screw, and a pair of tine members extending diagonally from the spacer end. The tines are constructed and arranged to penetrate adjacent bone, providing supplemental fixation and resisting migration or rotational forces of the implant. The staple member may be flexible to accommodate insertion while still maintaining anchoring engagement once implanted.

An objective of the present invention is to provide a fixation device with a malleable structure capable of conforming to various spinal joint spacings.

A further objective of the present invention is to provide a fixation device including a locking member that prevents bone screws from backing out once implanted.

Another objective of the present invention is to provide a fixation device that can be inserted with unobstructed access angles due to the accordion-like side members.

Yet another objective of the present invention is to provide a fixation device including a staple member at the spacer end, the staple member having tines adapted to penetrate adjacent bone and configured to resist migration and rotational forces of the implant.

Other objectives and advantages of this invention will become apparent from the following description taken in conjunction with any accompanying drawings wherein are set forth, by way of illustration and example, certain embodiments of this invention. Any drawings contained herein constitute a part of this specification and include exemplary embodiments of the present invention and illustrate various objects and features thereof.

### BRIEF DESCRIPTION OF THE FIGURES

FIG. 1 is a front view of a locking fixation device;

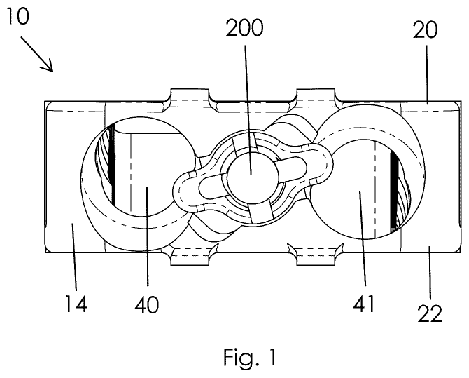

FIG. 2 is a front view thereof;

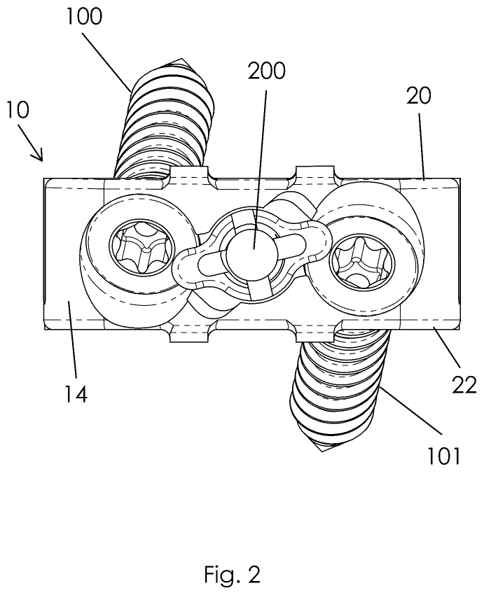

FIG. 3 is a side view thereof;

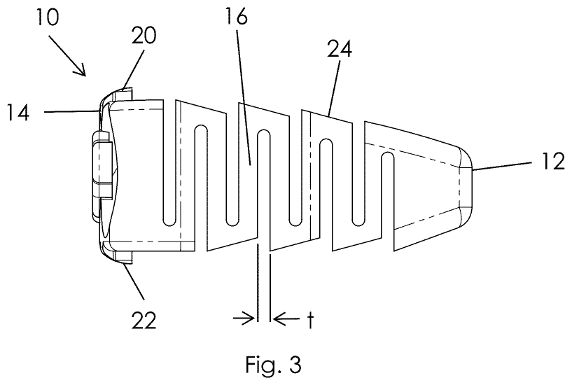

FIG. 4 is a side view thereof;

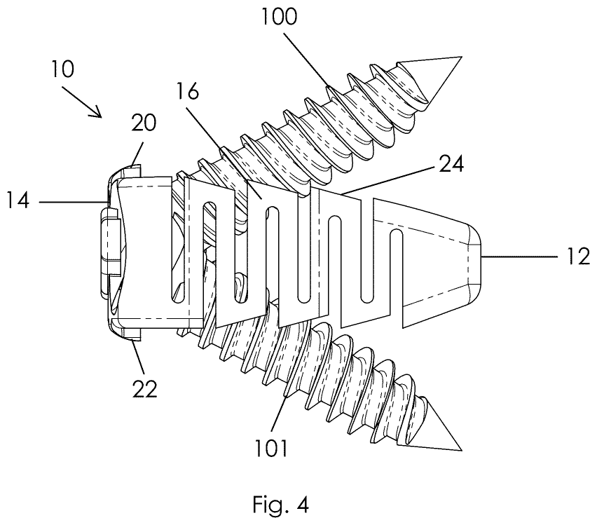

FIG. 5 is a top view thereof;

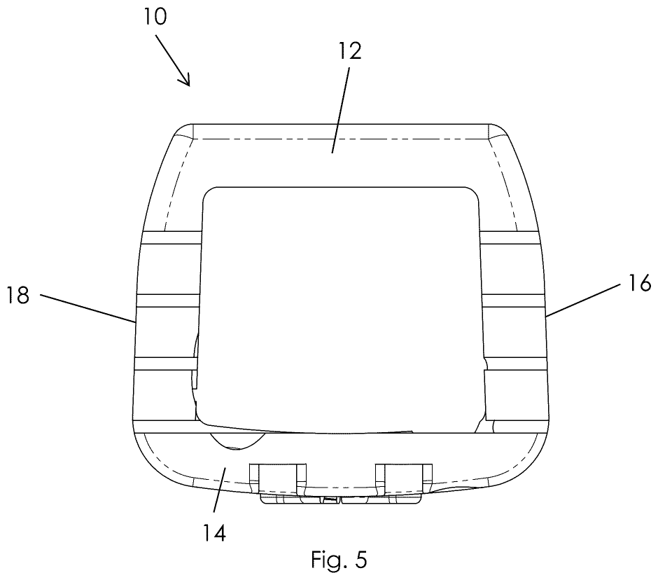

FIG. 6 is a top view thereof with bone screws;

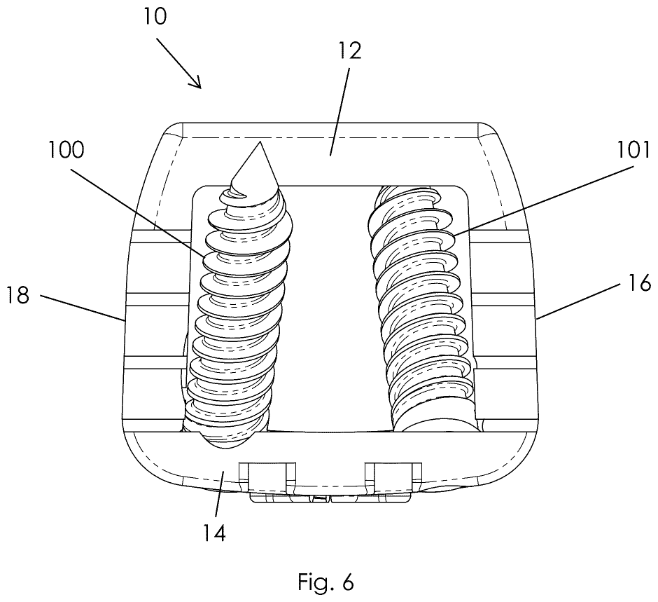

FIG. 7 is a perspective view thereof;

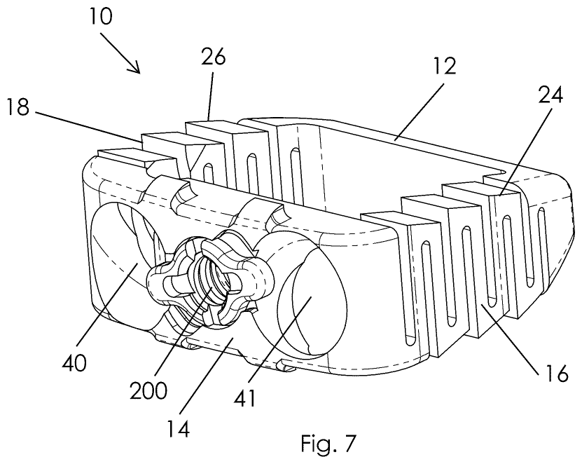

FIG. 8 is a perspective view thereof;

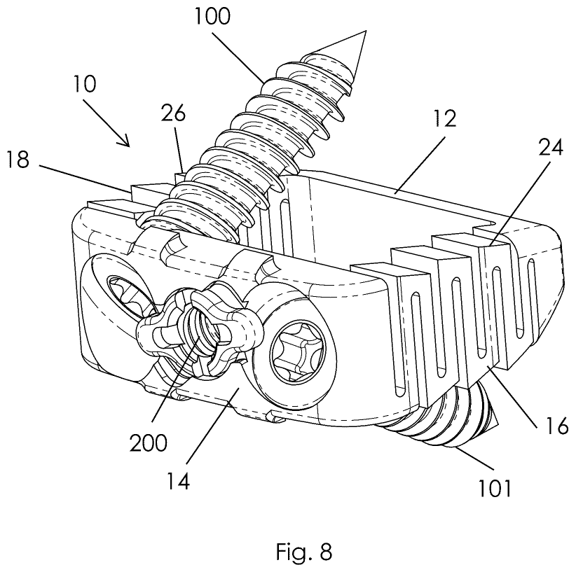

FIG. 9 is an exploded view thereof;

FIG. 10 is a detailed view of the fixation device showing the locking mechanism;

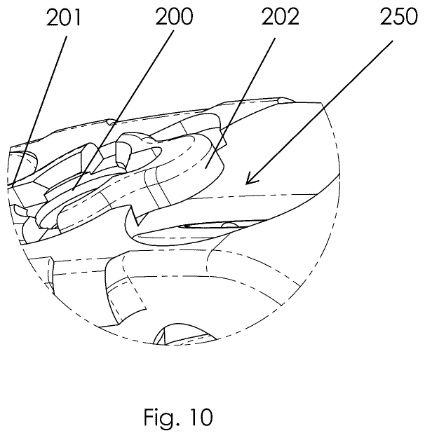

FIG. 11 is a cross-sectional view thereof;

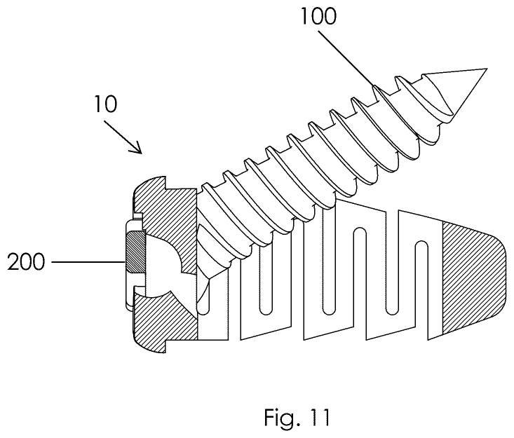

FIG. 12 is a detailed view thereof illustrating a locking mechanism to retain a bone screw from backing out;

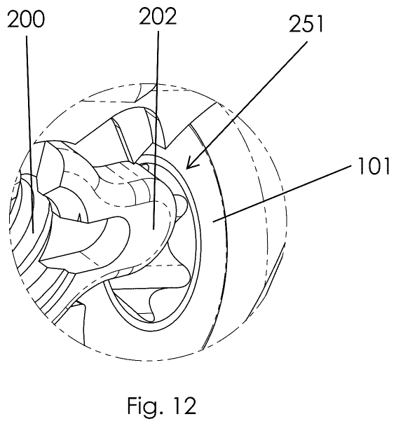

FIG. 13 is a top perspective view of a locking fixation device, including a staple;

FIG. 14 is a rear perspective view thereof;

FIG. 15 is an exploded view thereof;

FIG. 16 is an exploded view thereof;

FIG. 17 is a side view thereof;

FIG. 18 is a top perspective view of the locking fixation device with bone screws;

FIG. 19 is a rear perspective view thereof;

FIG. 20 is an exploded view thereof;

FIG. 21 is an exploded view thereof;

FIG. 22 is a side view thereof;

FIG. 23 is a front perspective view of the locking fixation device with an offset staple;

FIG. 24 is a rear perspective view thereof;

FIG. 25 is an exploded view thereof;

FIG. 26 is an exploded view thereof; and

FIG. 27 is a side view thereof.

### DETAILED DESCRIPTION OF THE INVENTION

It should be understood that numerous specific details, relationships, and methods are set forth to provide a full understanding of the invention. One having ordinary skill in the relevant art, however, will readily recognize that the invention can be practiced without one or more of the specific details or with other methods. The present invention is not limited by the illustrated ordering of acts or events, as some acts may occur in different orders and/or concurrently with other acts or events. Furthermore, not all illustrated acts or events are required to implement a methodology in accordance with the present invention.

The following description of the preferred embodiments is merely exemplary in nature and is in no way intended to limit the invention, its application or uses.

Referring now to the Figures in general, disclosed is a facet joint fixation device that consists of a malleable wedge-shaped implant 10 that can provide anchoring for a bone screw implant. The facet fixation device is preferably constructed from a single piece of biocompatible materials, such as titanium, or any conventional material used for surgical implants, such as stainless steel and its many different alloys, titanium alloys, metallic alloys, polymeric materials, plastics, plastic composites, ceramic and any other metal or material with the requisite strength and biologically inert properties. However, it is to be understood, that the various parts of the device may be constructed from various materials in some embodiments. For example, the side members may be made from a material that provides the requisite strength but also malleability, whereas the insert end and spacer end are made from a rigid material that can be subjected to force without bending or buckling. If desired, the insert end and/or the side members can be coated with a lubricant or material that provides a lubricating effect. In some embodiments, the insert end and/or the spacer end may comprise one or more layers of materials, such as, for example, plastic, polymers, metals or any other biocompatible conventional material(s). In other embodiments, the device may be coated with a biocompatible material, for example, medical grade thermoplastic elastomeric compounds.

The malleable wedge-shaped implant 10 is defined by an insert end 12 spaced apart from an outer or spacer end 14 by a first 16 and second 18 side members forming an accordion like shape having a thickness. Each side member is further defined as a continuous piece of material having a first 16 side surface with a first series of recesses 24 forming a sinusoidal pattern extending therefrom and perpendicular thereto. The recesses 24, 26 can be any shape and size as long as they provide flexibility to the side members. In addition, the recesses 24, 26 serve the purpose of stabilizing or provide grip to prevent slippage or movement of the device. Each side member 16 and 18 is capable of being bent back and forth due to the retained thickness which provide spring hinge type resilience. The first 16 side surface has a slope between adjacent recesses 24, and a second series of recesses 26 extend from a second 18 side surface positioned adjacent to the first series of recesses 24 with the second 18 side surface having a slope between adjacent recesses 26. The thickness of the side member 16, 18 is uniform between the spacer end 14 and the insert end 12.

In preferred embodiments, the side members can flex at least about 20° relative to a horizontal axis, preferably about 45° relative to a horizontal axis, preferably about 50° relative to a horizontal axis, preferably about 75° relative to a horizontal axis, preferably 90° relative to a horizontal axis, preferably about 180° relative to a horizontal axis. In some embodiments, the flexibility is about 270° relative to a horizontal axis.

The fixation device 10 includes a first receiving hole 40 extending through the spacer end 14 from an upper surface 20 through a lower surface 22. The first receiving hole 40 is adapted to receive a first bone screw 100 for securing to a vertebral body. Likewise, the fixation device 10 includes a second receiving hole 41 extending through the spacer end 14 from an upper surface 20 through a lower surface 22. The second receiving hole 41 is adapted to receive a second bone screw 101 for securing to an adjacent vertebral body. In an alternative embodiment, the first receiving hole 40 is formed at a first angle and the second receiving hole 41 is formed at a second angle traverse to the first angle.

To ensure that either bone screw 100, 101 does not reverse direction and remains in place after inserted between two vertebral bodies, a locking member 200 is rotatably secured to the spacer end 14 positioned between the first 40 and second 41 receiving hole. The locking member 200 includes tabs 201, 202 constructed and arranged to rotate from an installation position 250 allowing insertion of said bone screws to a locking position 251 to prohibit bone screw movement. In a preferred embodiment, the locking member 200 includes a base 204 that has a raised section elevating the locking member 200 into the installation position 250. Rotation of the locking member 200 from the installation position 250 to the locking position 251 bias the locking member 200 in the locking position 251 wherein each the tabs 201, 202 are over the tops of the bone screws 100, 101.

Accordingly, in preferred embodiments, a facet spacer device 10 comprises an insert end 12, a spacer end 14, wherein the insert end 12 and the spacer end 14 are connected by a first 16 and a second 18 side member, wherein each side member 16, 18 comprises at least one recess 24, 26 defining a hinge structure. In a preferred embodiment, the side members 16, 18 comprise a plurality of recesses 24, 26 defining a hinge structure forming a sinusoidal pattern extending therefrom and perpendicular thereto.

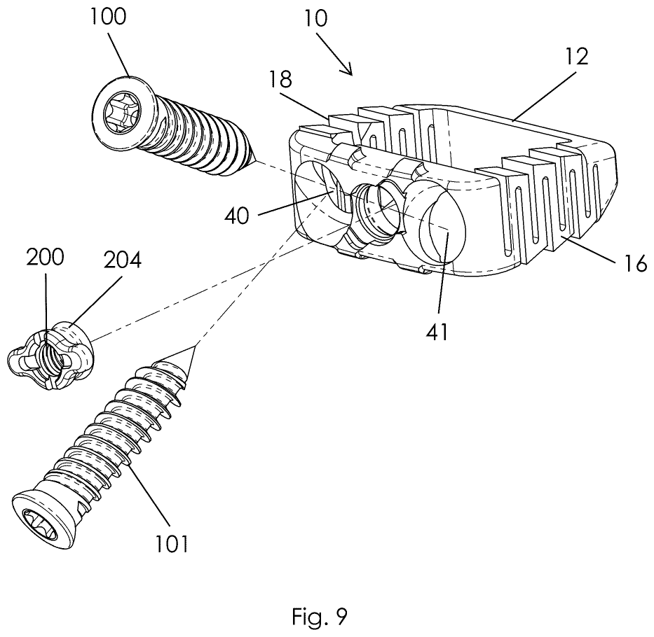

In a preferred embodiment, the insert end 12 comprises a tapered end and the spacer end 14 comprises a curved surface or a planar surface having flat or curved sides or combinations thereof, and at least one receiving hole 40, 41 for receiving a bone screw 100, 101 or other instruments. In a preferred embodiment, the fixation device 10 includes a first receiving hole 40 adapted to receive a first bone screw 100 and a second receiving hole 41 adapted to receive a second bone screw 101 to secure two vertebral bodies, as shown in FIG. 9.

In preferred embodiments, the spacer end 14 is wider than the height of the side members 16, 18. The side members 16, 18 connecting the insert end 12 and the spacer end 14 are attached to the spacer end 14, in a position equidistant from each end of the planar or curved surface. Accordingly, the height of the spacer end 14 is greater than the height of the side members 16, 18 by at least about 0.1 fold to about 10 fold.

Embodiments are also directed to methods and procedures for using the device. In a preferred embodiment, a method of implanting a bone screw in a patient in need thereof, comprises inserting a malleable wedge-shaped implant between adjacent vertebrae, the malleable wedge-shaped implant comprising an insert end, an outer end, wherein the insert end and the outer end are spaced apart by a first and a second side member, each side member having one or more recesses forming an accordion like shape having a thickness, defined as a continuous piece of material having a first side surface with a first series of recesses forming a sinusoidal pattern extending therefrom and perpendicular thereto. The insert end comprises a tapered end for ease of inserting the device in between adjacent vertebrae.

In preferred embodiments, the outer end comprises a planar surface having flat or curved sides or combinations thereof, and at least one receiving hole for receiving the bone screw. It is to be understood that the openings may vary in size for receiving various surgical or other instruments.

In embodiments, the recesses defining the accordion like shaped structure further provide an anchoring thereby preventing the device from moving during the procedure or bone screw pullout.

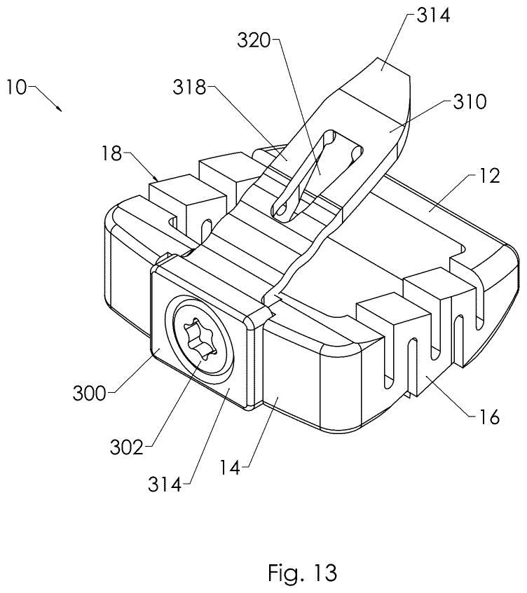

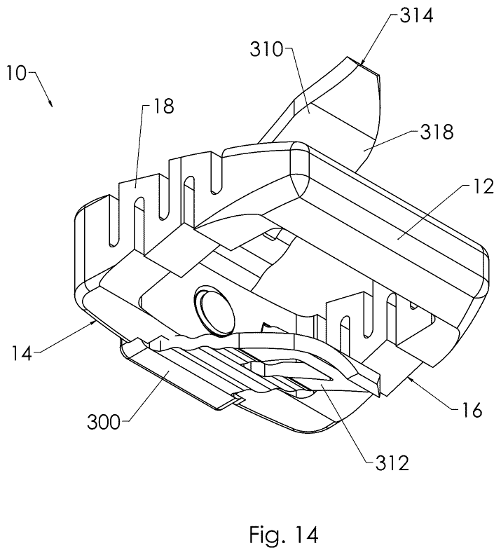

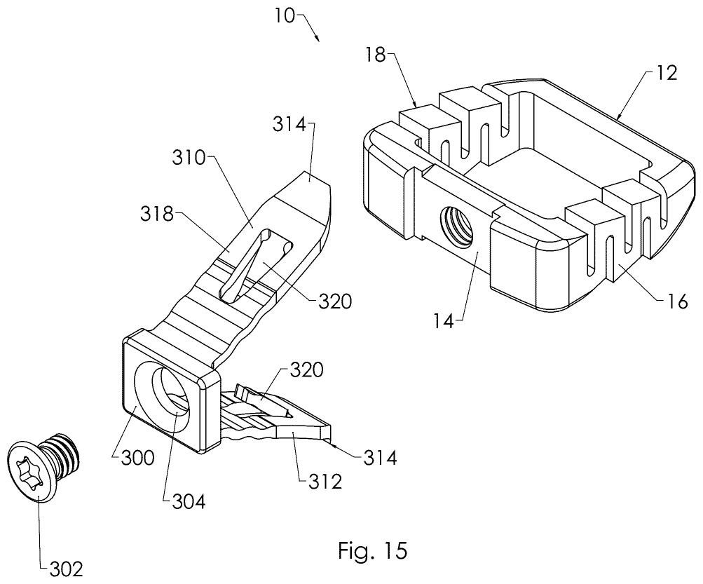

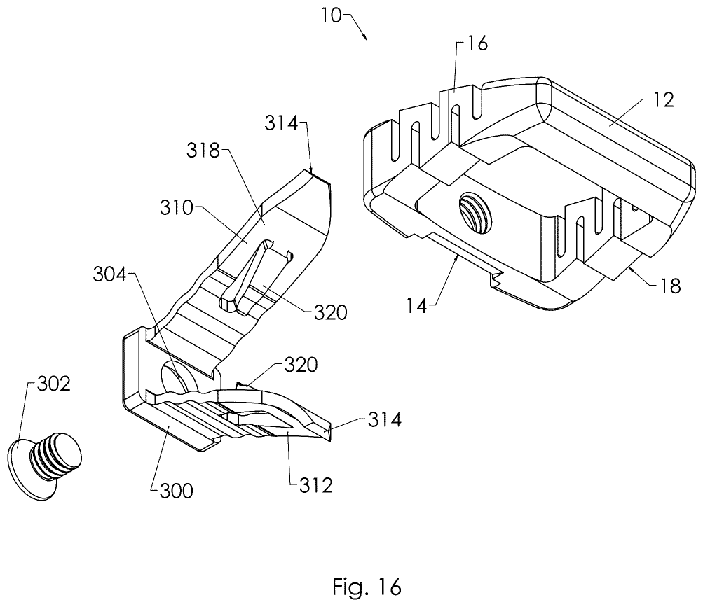

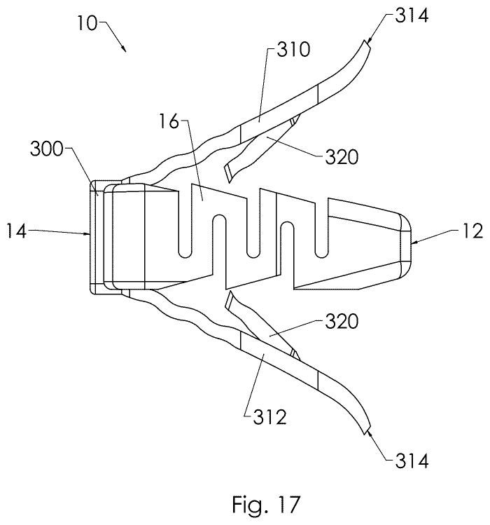

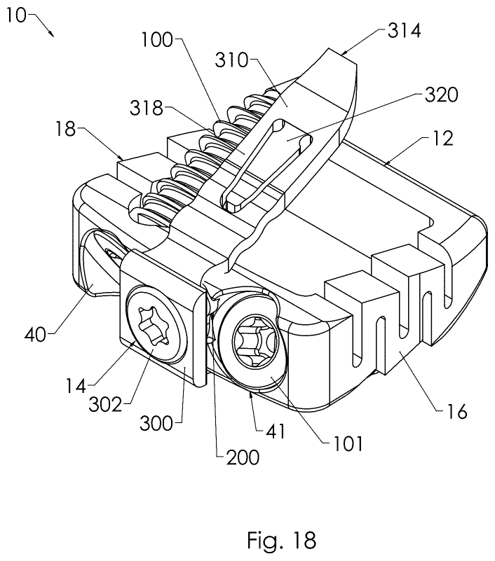

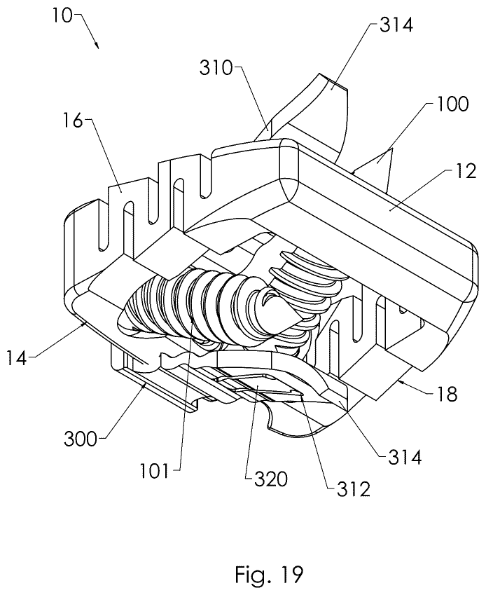

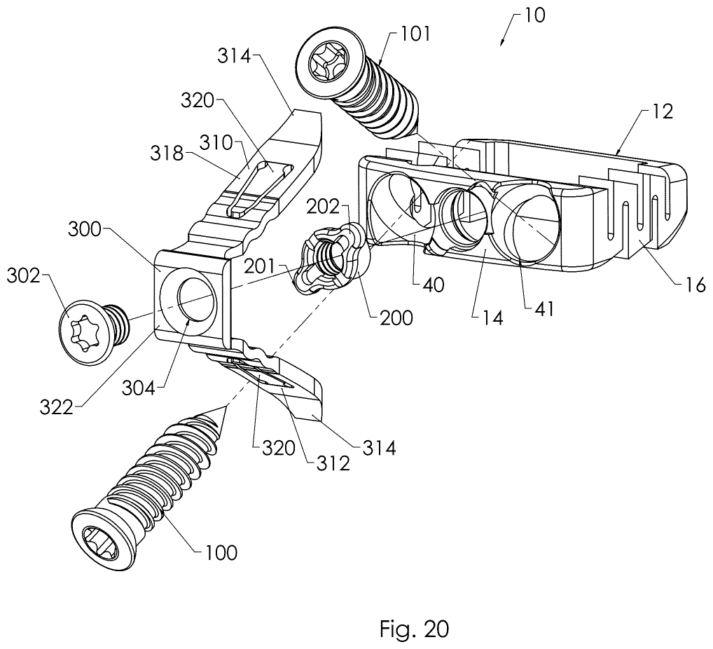

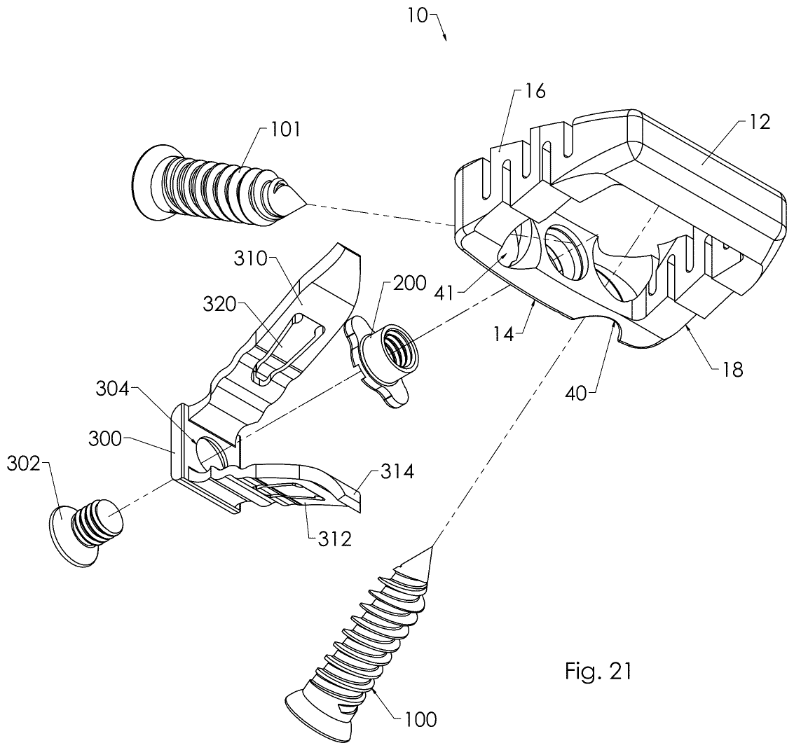

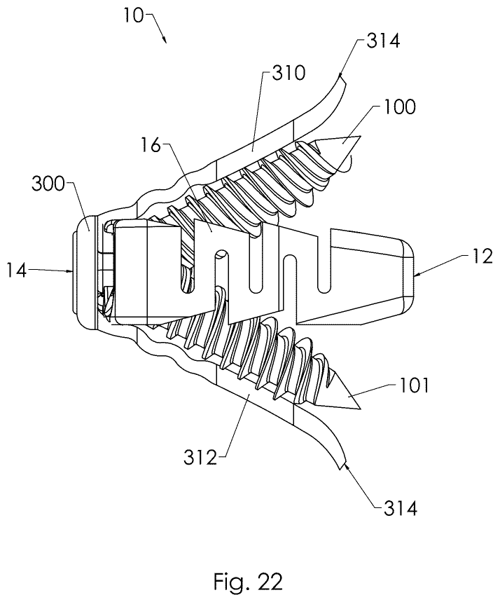

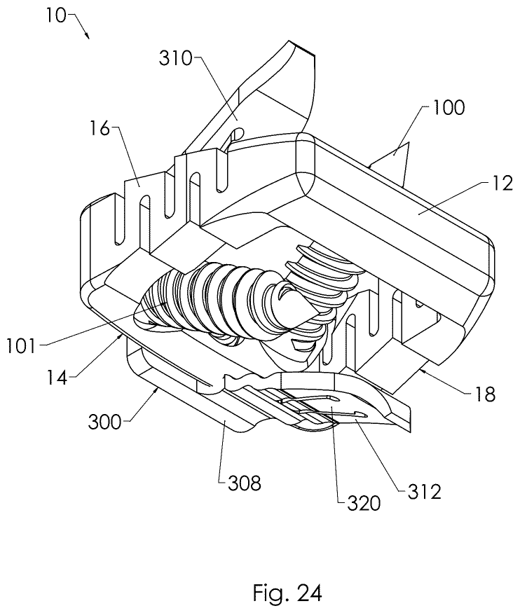

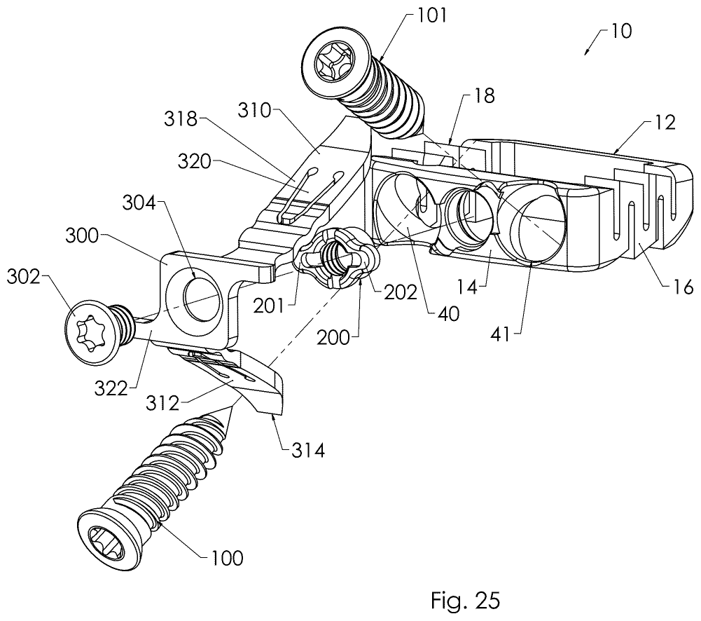

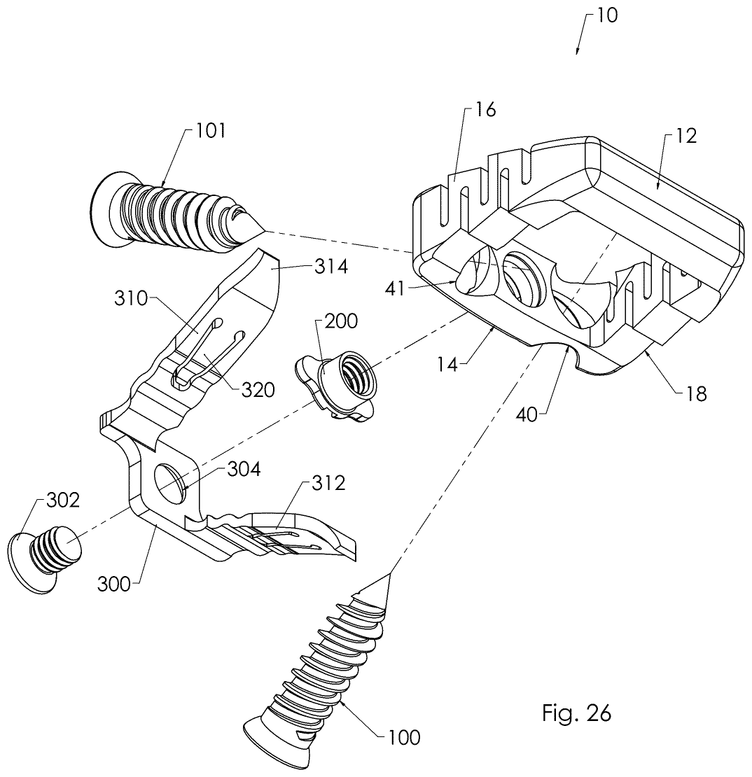

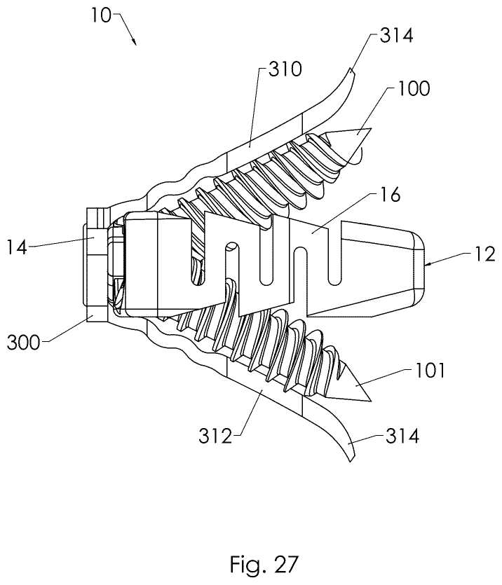

In additional embodiments, as illustrated in FIGS. 13-27, the fixation device 10 further includes a staple member 300 coupled to the spacer end 14. The staple member 300 is disposed between a locking screw 302 and the locking member 200. The staple member 300 includes an aperture 304 through which the locking screw 302 passes, the locking screw 302 being threadably engaged with the locking member 200 to secure the staple member 300 to the spacer end 14. The staple member 300 further includes a pair of tines 310, 312 having an extruded body 318 extending diagonally from the spacer end 14.

The tines 310, 312 are constructed and arranged to penetrate bone and provide anchoring engagement. Once implanted, the tines 310, 312 resist migration or rotation of the fixation device 10 relative to the surrounding bone structures. In some embodiments, the tines 310, 312 may be oriented to diverge from one another to increase surface contact within the bone.

In some embodiments, the tines 310, 312 are flexible to permit deflection during insertion. The flexibility of the tines 310, 312 allows them to deform as the staple member 300 is driven into bone and thereafter return toward their original position to maintain anchoring engagement once implanted.

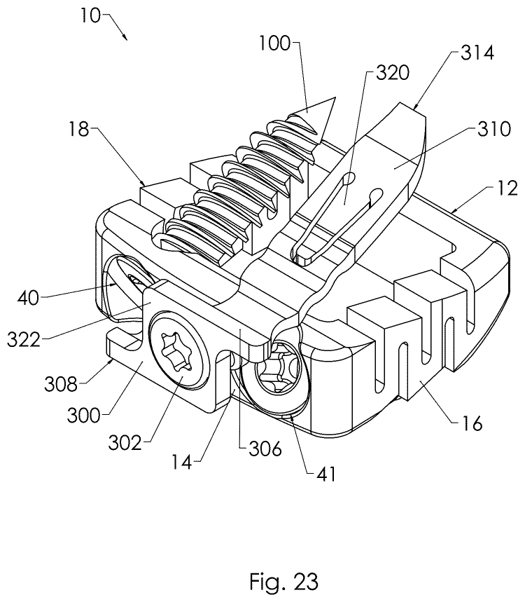

In an alternative embodiment, shown for example in FIG. 23, the tines 310, 312 are offset from one another along the spacer end 14. In this embodiment, one tine 310 originates from a first lateral side 306 of the staple base 322 and the other tine 312 originates from an opposite lateral side 308. The offset configuration permits staggered engagement with the bone adjacent the first and second receiving holes 40, 41, thereby providing additional stabilization.

In some embodiments, the tines 310, 312 include one or more integral tabs 320 disposed along the body of the tines. The tabs 320 may be formed integrally with the tine material and configured to lie generally flush with the tine body 318 prior to insertion. Upon deflection of the tines 310, 312 during insertion, the tabs 320 may project outwardly to engage surrounding bone. The tabs 320 thereby resist withdrawal of the tines 310, 312 from bone and contribute to the overall anchoring stability of the fixation device 10.

In some embodiments, the distal ends 314 of the tines 310, 312 are curved relative to the longitudinal axis of the tine body. The curved distal ends 316 may facilitate insertion of the tines into bone and may further provide resistance to withdrawal once implanted by engaging surrounding bone tissue. In certain embodiments, the curvature also distributes insertional forces along the tine body, thereby reducing stress concentration at the tip of the tine.

Although particular embodiments of the staple member 300 have been illustrated and described, it is to be understood that the present invention is not limited to the specific structural arrangements shown. Various modifications may be made without departing from the scope of the invention. For example, the tines 310, 312 may vary in length, angle, or cross-sectional shape, and the number or configuration of tabs 320 may differ depending on surgical preference or bone quality.

The staple member 300 may be fabricated from the same biocompatible materials described above for the implant body, including metals, metal alloys, polymers, and composite materials. In some embodiments, the staple member 300 may be formed as a single piece with the implant body, while in other embodiments the staple member 300 may be a separate component coupled to the spacer end 14 during implantation.

The term “coupled” is defined as connected, although not necessarily directly, and not necessarily mechanically. The use of the word “a” or “an” when used in conjunction with the term “comprising” in the claims and/or the specification may mean “one,” but it is also consistent with the meaning of “one or more” or “at least one.” The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a method or device that “comprises,” “has,” “includes” or “contains” one or more steps or elements, possesses those one or more steps or elements, but is not limited to possessing only those one or more elements. Likewise, a step of a method or an element of a device that “comprises,” “has,” “includes” or “contains” one or more features, possesses those one or more features, but is not limited to possessing only those one or more features.

The terms “comprise” (and any form of comprise, such as “comprises” and “comprising”), “have” (and any form of have, such as “has” and “having”), “include” (and any form of include, such as “includes” and “including”), and “contain” (and any form of contain, such as “contains” and “containing”) are open-ended linking verbs. As a result, a method or device that “comprises,” “has,” “includes,” or “contains” one or more steps or elements, possesses those one or more steps or elements, but is not limited to possessing only those one or more elements. Likewise, a step of a method or an element of a device that “comprises,” “has,” “includes,” or “contains” one or more features, possesses those one or more features, but is not limited to possessing only those one or more features. Furthermore, a device or structure that is configured in a certain way is configured in at least that way, but may also be configured in ways that are not listed.

It is to be understood that while a certain form of the invention is illustrated it is not to be limited to the specific form or arrangement herein described and shown. It will be apparent to those skilled in the art that various changes may be made without departing from the scope of the invention, and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary, and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A fixation device adapted to be applied to the human spine for fusing vertebral bodies together, said fixation device comprising:
a fixation body having an insert end spaced apart from a spacer end by a side member forming an accordion like shape having a thickness, said side member further defined as a continuous piece of material having a first side surface with a first series of recesses forming a sinusoidal pattern extending therefrom and perpendicular thereto, said first side surface having a slope between adjacent recesses, and a second series of recesses extending from a second side surface positioned adjacent said first series of recesses with said second side surface having a slope between adjacent recesses, said thickness of said side member is uniform between said spacer end and said insert end;
a first receiving hole extending through said spacer end from an upper surface through a lower surface, said first receiving hole being adapted to receive a first bone screw for securing to a vertebral body;
a second receiving hole extending through said spacer end from an upper surface through a lower surface, said second receiving hole being adapted to receive a second bone screw for securing to an adjacent vertebral body;
a locking member rotatably secured to said spacer end positioned between said first and second receiving hole, said locking member having tabs constructed and arranged to rotate from an installation position allowing insertion of said bone screws and a locking position to prohibit bone screw movement; and
a staple member coupled to said spacer end and disposed between a locking screw and said locking member, said staple member comprising an aperture through which the locking screw passes, the staple member further including a pair of tines having an extruded body extending diagonally from the spacer end, wherein the locking screw is threadably engaged with the locking member to secure the staple member to the spacer end.

2. The fixation device of claim 1, wherein said first receiving hole is formed at a first angle and said second receiving hole is formed at a second angle traverse to said first angle.

3. The fixation device of claim 1 wherein a base of said locking member has a raised section elevating said locking member in said installation position.

4. The fixation device of claim 3 wherein rotation of said locking member from said installation position to said locking position bias said locking member in the locking position wherein each said tab is over the top of a bone screw.

5. The fixation device of claim 1, wherein the insert end comprises a tapered end.

6. The fixation device of claim 1, wherein the spacer end is wider than the height of the side members by at least about 0.1 fold to about 10 fold.

7. The fixation device of claim 1, wherein the insert end and the spacer end are rigid.

8. The fixation device of claim 1, wherein said tines are constructed and arranged to penetrate bone and resist migration or rotation of the fixation device.

9. The fixation device of claim 1, wherein said tines are flexible.

10. The fixation device of claim 1, wherein said staple member comprises a pair of tines that are offset from one another along said spacer end.

11. The fixation device of claim 1, wherein said tines comprise one or more integral tabs disposed along the body of said tine, said tabs being configured to engage bone and resist withdrawal when said tines are flexed during insertion.

12. A fixation device adapted to be applied to the human spine for fusing vertebral bodies together, said fixation device comprising:
a fixation body having an insert end spaced apart from a spacer end by a side member forming an accordion-like shape having a thickness, said side member further defined as a continuous piece of material having a first side surface with a first series of recesses forming a sinusoidal pattern extending therefrom and perpendicular thereto, said first side surface having a slope between adjacent recesses, and a second series of recesses extending from a second side surface positioned adjacent said first series of recesses with said second side surface having a slope between adjacent recesses, said thickness of said side member being uniform between said spacer end and said insert end;
a first receiving hole extending through said spacer end from an upper surface through a lower surface, said first receiving hole being adapted to receive a first bone screw for securing to a vertebral body, wherein said first receiving hole is formed at a first angle;
a second receiving hole extending through said spacer end from an upper surface through a lower surface, said second receiving hole being adapted to receive a second bone screw for securing to an adjacent vertebral body, wherein said second receiving hole is formed at a second angle transverse to said first angle;
a locking member rotatably secured to said spacer end positioned between said first and second receiving hole, said locking member having tabs constructed and arranged to rotate from an installation position allowing insertion of said bone screws and a locking position to prohibit bone screw movement, wherein a base of said locking member has a raised section elevating said locking member in said installation position; and
a staple member coupled to said spacer end and disposed between a locking screw and said locking member, said staple member comprising an aperture through which the locking screw passes, said staple member further including a pair of flexible tines extending diagonally from the spacer end, wherein the locking screw is threadably engaged with the locking member to secure the staple member to the spacer end.

13. The fixation device of claim 12, wherein rotation of said locking member from said installation position to said locking position biases said locking member in the locking position wherein each said tab is over the top of a bone screw.

14. The fixation device of claim 12, wherein the insert end comprises a tapered end.

15. The fixation device of claim 12, wherein the spacer end is wider than the height of the side members by at least about 0.1 fold to about 10 fold.

16. The fixation device of claim 12, wherein the insert end and the spacer end are rigid.

17. The fixation device of claim 12, wherein said tines are constructed and arranged to penetrate bone and resist migration or rotation of the fixation device.

18. The fixation device of claim 12, wherein said staple member comprises a pair of tines that are offset from one another along said spacer end.

19. The fixation device of claim 12, wherein said tines comprise one or more integral tabs disposed along the body of said tine, said tabs being configured to engage bone and resist withdrawal when said tines are flexed during insertion.

20. The fixation device of claim 12, wherein distal ends of said tines are curved relative to the tine body to facilitate insertion into bone and resist withdrawal once implanted.
